(19) United States
(12) Patent Application Publication
(10) Pub. No.: US 2019/0216722 A1
(43) Pub. Date: Jul. 18, 2019

(54) METHODS TO TREAT HEART FAILURE

(71) Applicant: Ghassan S. Kassab, La Jolla, CA (US)

(72) Inventor: Ghassan S. Kassab, La Jolla, CA (US)

(21) Appl. No.: 16/247,238

(22) Filed: Jan. 14, 2019

Related U.S. Application Data
(60) Provisional application No. 62/617,209, filed on Jan. 13, 2018.

(51) Int. Cl. A61K 9/00; A61K 38/43; A61K 38/48

(52) U.S. Cl. CPC A61K 9/0019; A61K 38/4886; A61K 38/43

(57) ABSTRACT

Methods to treat heart failure. A method described herein includes injecting a material comprising an enzyme into a left ventricle of a cardiac tissue, the material targeting an extracellular matrix of the cardiac tissue to treat heart failure with preserved injection fraction (HFpEF).

### PRIORITY

The present patent application is related to, and claims the priority benefit of, U.S. Provisional Patent Application Ser. No. 62/617,209, filed on Jan. 13, 2018, the contents of which are hereby incorporated by reference in their entirety into this disclosure.

### BACKGROUND

The role of the extracellular matrix (ECM) in heart health and disease is largely unknown. The ECM is a network of protein fibers in all tissues, including the heart, which can store and transmit information (mechanotransduction) at different time scales—well past the life span of many individual cells. Knowledge about how the ECM instructs cells to behave and how it stores long-term memory may change the way we think of and treat cardiovascular disease, making it a crucial frontier topic in heart research.

Because heart failure with preserved ejection fraction (HFpEF) has proven particularly challenging to treat, it is perhaps not surprising that we have yet to find an evidence-based HFpEF treatment beyond diuretics for fluid overload, and conventional pharmaceutical treatments for co-morbidities. Experimental and computational studies have shown that injection treatment can be beneficial for patients with heart failure (HF) (Lee et al., 2015, Mann et al., 2016, Wall et al., 2006, Wenk et al., 2009, Lee et al., 2013). Approximately 50% of patients with HF have HFpEF (Bursi et al., 2006, Owan et al., 2006). HFpEF is one of the life-threatening diseases for which optimal treatments remain controversial (Vasan et al., 1995, Bhuiyan and Maurer, 2011). On the other hand, the population of patients with HFpEF has increased in the past, and it will continue to increase (Benjamin et al., 2017, Heidenreich et al., 2013, Liu et al., 2013, Steiberg et al., 2012). HFpEF could cause end stage HF for which the current treatment options are extremely limited; namely, mechanical circulatory support devices, or heart transplantation. These treatment options, if available, are risky and expensive.

### BRIEF SUMMARY

The present disclosure includes disclosure of using a minimally invasive injection of collagenase at strategic locations in the left ventricle (LV) that has an abnormally stiff extracellular matrix as an effective treatment for heart failure with preserved ejection fraction (HFpEF). As referenced in further detail herein, physics-based finite element models of the LV were created from LV geometry and pressures recorded during experiments conducted on one swine at a base-line stage where the LV was normal, and six weeks following LV pressure overload. The collagenase injection treatment was simulated using a set of 1-to-16 injections in the LV free wall with or without 12 injections in the septum. The stiffness of injections was also altered. Two types of injections were considered: (1) spherical-shaped, in which the injected regions were shaped like spheres centered at the injection site, and (2) cylindrical-shaped, in which the injected regions were shaped like cylinders spreading in the transmural direction from endocardium to epicardium. Three transmural locations for spherical injections were used: mid-wall, epicardium and endocardium. The stiffness, pattern, position, and volume of injections played key roles in the outcomes of injection treatment. At an end diastolic pressure of 23 mmHg, when 8.0 ml of the free wall was covered by cylindrical injections, end diastolic volume (EDV) increased by 15.0%, whereas an increase up to 11.0 ml due to injections in the septum and free wall led to a 26.0% increase in the EDV. Although the endocardial injections had a lower volume, they led to a higher EDV (43.8 ml) compared to injections in mid-wall (43.7 ml) and epicardium (41.2 ml). Additionally, the end diastolic pressure-volume relation shifted toward larger EDVs with injections. Endocardial regions did not experience noticeably high distortions due to injections. Using finite element modeling, the optimal injections can be effectively planned for injection treatment, which is an effective option for improving LV EDV in HFpEF.

The present disclosure includes disclosure of using a minimally invasive injection of collagenase at strategic locations in the left ventricle (LV) that has an abnormally stiff extracellular matrix as an effective treatment for heart failure with preserved ejection fraction (HFpEF).

The present disclosure includes disclosure of injecting colleagenase to treat HFpEF. The present disclosure includes disclosure of injecting one or more enzymes that target proteoglycans and ground substance to treat HFpEF.

The present disclosure includes disclosure of a method, comprising the step of injecting a material into a cardiac tissue that targets an extracellular matrix of the cardiac tissue to treat heart failure.

The present disclosure includes disclosure of a method, wherein the material comprises an enzyme, and wherein the injecting step is performed to inject the enzyme into the cardiac tissue.

The present disclosure includes disclosure of a method, wherein the cardiac tissue comprises a left ventricle wall, and wherein the injecting step is performed to inject the enzyme into the left ventricle wall.

The present disclosure includes disclosure of a method, wherein the enzyme comprises collagenase, and wherein the injecting step is performed to inject the collagenase into the cardiac tissue.

The present disclosure includes disclosure of a method, wherein the enzyme comprises at least one enzyme that targets proteoglycans and ground substance, and wherein the injecting step is performed to inject the at least one enzyme that targets proteoglycans and ground substance into the cardiac tissue.

The present disclosure includes disclosure of a method, wherein the extracellular matrix is stiff, and wherein the injecting step is performed to inject the material into the cardiac tissue that targets the stiff extracellular matrix.

The present disclosure includes disclosure of a method, wherein the step of injecting reduces a stiffness of the extracellular matrix of the cardiac tissue.

The present disclosure includes disclosure of a method, wherein the step of injecting reduces a stiffness of a diastolic myocardium of the cardiac tissue.

The present disclosure includes disclosure of a method, wherein the heart failure is heart failure with preserved injection fraction (HFpEF), and wherein the step of injecting is performed to treat HFpEF.

The present disclosure includes disclosure of a method, wherein the step of injecting is performed so that material injected into the cardiac tissue has a spherical shape.

The present disclosure includes disclosure of a method, wherein the step of injecting is performed so that material injected into the cardiac tissue has a cylindrical shape.

The present disclosure includes disclosure of a method, wherein the step of injecting is performed so that the cylindrical shape of the material injected into the cardiac tissue extends between an endocardium and an epicardium of the cardiac tissue.

The present disclosure includes disclosure of a method, wherein the step of injecting is performed to inject the material at a first injection site and at a second injection site of the cardiac tissue.

The present disclosure includes disclosure of a method, wherein the first injection site is selected from the group consisting of a mid-wall, an epicardium, and an endocardium.

The present disclosure includes disclosure of a method, wherein the step of injecting is performed to inject between 7.0 mL and 11.8 mL of the material into the cardiac tissue.

The present disclosure includes disclosure of a method, comprising the step of injecting a material comprising an enzyme into a left ventricle of a cardiac tissue, the material targeting an extracellular matrix of the cardiac tissue to treat heart failure with preserved injection fraction (HFpEF).

The present disclosure includes disclosure of a method, wherein the step of injecting is performed so to inject the material between once and sixteen times into a free wall of the left ventricle.

The present disclosure includes disclosure of a method, further comprising the step of injecting the material into a septum of the cardiac tissue between once and twelve times.

The present disclosure includes disclosure of a method of using a minimally invasive injection of an extracellular matrix targeting material at a location in a cardiac tissue having an abnormally stiff extracellular matrix as an effective treatment for heart failure with preserved injection fraction (HFpEF), the method comprising the steps of percutaneously accessing a pericardial space of a heart, and injecting a free wall of the cardiac tissue with the extracellular matrix targeting material, the extracellular matrix targeting material comprising collagenase or another enzyme that targets proteoglycans and ground substance.

The present disclosure includes disclosure of a method, further comprising the steps of first determining an end diastolic volume (EDV) of the heart before the step of injecting is performed; and second determining an end diastolic volume (EDV) of the heart after the step of injecting is performed, wherein the EDV of the heart after the step of injecting is higher than the EDV of the heart before the step of injecting.

The present disclosure includes disclosure of a method, further comprising the step of injecting a septum with an extracellular matrix targeting material.

The present disclosure includes disclosure of a method, further comprising the step of moving the needle while injecting the cardiac tissue.

The present disclosure includes disclosure of a method, further comprising the step of using finite element modeling to determine ideal injection parameters of stiffness, pattern, position and volume in order to maximize the effectiveness of the treatment,

The present disclosure includes disclosure of a method, wherein the extracellular matrix targeting material is collagenase.

The present disclosure includes disclosure of a method, wherein the extracellular matrix targeting material is one or more enzymes that target proteoglycans and ground substance.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1: Site, shape and stiffness of injections made in the LV free wall. The free wall injections were made in a 4×4 pattern in the circumferential and longitudinal directions. A set of 12 injections was also made in the septum. Two shapes of injections were used: spherical (left) and cylindrical (right). For the injections in the free wall, three transmural locations for injections were used: epicardium, mid-wall and endocardium. In the nodes located within 5 mm from the injection center, C_{index}=0, and for nodes located between 5 and 10 mm, C_{index} changed linearly with distance. This is a three dimensional view with the free wall on the left side.

FIG. 2: EDV vs. injection volume and injection stiffness. The EDV increased with the volume of injections. The higher the stiffness of the injected tissue, the lower the EDV. Spherical injections were used for this surface. The color gradient shows changes in EDV with injection volume and stiffness scale.

FIG. 3: End systolic strain distribution in the myofiber direction. The strains in the myofiber direction were altered by injections. The results pertain to end systole for 16 cylindrical and 16 spherical-injections (P_{scaling}=0.01). Injection volumes are summarized in Table (1). This is a long-axis view with the cut plane as shown.

FIG. 4: Injections in the septum and free wall led to higher diastolic LV volumes at all diastolic LV pressures compared to injections only in the free wall with either spherical or cylindrical injections. The triangle shows EDV, EDP. To plot these curves, EDV and EDP (small triangle) were calculated using FE modeling (P_{scaling}=0.01), after which the analytical formula EDP=αEDV^{β} (Klotz et al., 2006) was used.

FIG. 5: The injection treatment shifted EDPVR toward higher EDVs. The LV PV curve altered in the treated case such that it recovered toward the base-line case. Cylindrical injections (P_{scaling}=0.01) altered the EDV and EDPVR more noticeably than spherical injections (P_{scaling}=0.01). EDPVR was created using formula EDO=αEDV^{β} (Klotz et al., 2006). Injection volumes are summarized in Table (1).

FIG. 6: ED strain distribution in the myofiber direction. The endocardial surface experienced high myofiber strains at the injection sites. The effects of injections were more noticeable after cylindrical injections, which had more injection volume, than after spherical injections. This is a top view, looking at the LV base.

FIG. 7. The injections can be delivered in the septum and subendocardial region using the transcatheter access device recently developed in our lab. A: Schematic. B: Suction fixation. C: Hollow lumen wire. D: Distal needle tip.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As referenced herein, the present disclosure provides for the use of minimally invasive injections of collagenase at strategic locations in the left ventricle (LV) having, an abnormally stiff ECM as being an effective treatment for HFpEF. As noted in a 1991 manuscript (Guccione et al, 1991), the mechanical properties of the ECM in the normal dog LV are non-linear and anisotropic (i.e., transversely isotropic with respect to the local muscle fiber or myofiber direction). In other words, the stiffness of the ECM increases as it is stretched, and the ECM is approximately three times stiffer in the myofiber direction than in a plane perpendicular or transverse to the myofiber direction, even when the heart muscle or myocardium is not contracting.

The present disclosure includes details regarding an aortic banding procedure, whereby, in a translational swine model of chronic LV pressure overload (due to aortic banding), cardiac catheterization, real-time 3D transesophageal echocardiography (RT3D-TEE) was used, and finite element (FE) modeling was performed to quantify the mechanical properties of the ECM before and six weeks after aortic banding. The LV FE model with the stiffest ECM was then used to simulate regional ECM “de-stiffening” due to collagenase injections with different patterns, as provided in further detail herein.

### Methods

In vivo data were obtained from a swine study in which the geometry and pressure data were obtained. In the animal experiments, LV remodeling due to supravalvular aortic banding was quantified at baseline (0 weeks) and 6 weeks after aortic banding (LV pressure overload), at multiple time points using transesophageal 3D echocardiography. These experiments were conducted on the same animal.

All animal experiments were performed in accordance with national and local ethical guidelines, including the Guide for the Care and Use of Laboratory Animals, the Public Health Service Policy on Humane Care and Use of Laboratory Animals, and the Animal Welfare Act, and an approved California Medical Innovations Institute IACUC protocol regarding the use of animals in research.

Sedation was induced in a Yorkshire swine with TKX (Telazol 10 mg/kg, Ketamine 5 mg/kg, and Xylazine 5 mg/kg, IM). The animal was maintained on surgical anesthesia with isoflurane (1-2%) and oxygen while on the ventilator. The ventrolateral neck, the chest, and the right inguinal area were shaved and scrubbed with nolvasan, alcohol, and betadine. A 5Fr introducer sheath was placed percutaneously in the right jugular vein to administer fluids and drugs. Heparin 100 IU/kg body weight was administered IV before further instrumentation. A 6Fr introducer sheath was placed percutaneously into the right femoral artery. A hockey-stick guiding catheter was advanced over a wire through the introducer sheath towards the left ventricle to measure pressure.

Lidocaine 2% (1-4 mg/min, IV) was administered. The chest was opened through a midline sternotomy and the heart cradled in the pericardial sac. The root of the ascending aorta was then dissected free from the pulmonary artery. A cable tie covered with tygon tubing was passed around the ascending aorta and cinched until the LV pressure was 35%-50% above the baseline state. The pericardium was closed using non-absorbable sutures, the chest was closed in four layers, and the animal was allowed to recover. The echocardiography images and LV pressure at the base-line state, and six weeks after aortic banding were used for computational modeling.

### Computational Modeling

The LV geometry at end systole was reconstructed from echocardiographic images, and then the geometry was meshed using TrueGrid (XYZ Scientific Applications Inc, Pleasant Hill, Calif. USA).

ABAQUS software was used for FE computations (Simulia, Providence, R.I., USA). The main structure of the model follows the model described in the Living Heart Project literature (Baillargeon et al., 2014, Baillargeon et al., 2015, Sack et al., 2016).

The constitutive equations for the material behavior are based on the fiber-reinforced model described in the literature (Holzapfel and Ogden, 2009, Göktepe et al., 2011). Briefly, the following strain energy function is used for calculation of passive tissue stress:

$${ψ}_{dev} = \frac{a}{2 b} {e}^{b ( {l}_{1} - 3 )} + {\sum }_{t = f , s} \frac{{a}_{i}}{2 {b}_{i}} \{ {e}^{{{b}_{i} ( {l}_{4 i} - 1 )}^{2}} - 1 \} + \frac{{a}_{f s}}{2 {b}_{f s}} \{ {e}^{{{b}_{f s} ( {l}_{s f s} )}^{2}} - 1 \}$$

$${ψ}_{v o l} = \frac{1}{D} ( \frac{{J}^{2} - 1}{2} - ln ( J ) )$$

Here, a and b are isotropic stiffness material parameters of the tissue. The parameters with subscripts f, s, and fs refer to material parameters associated with stiffness in the fiber direction, sheet direction, and the connection between fiber and sheet directions. The invariants, l₁, l₄ᵢ and l_{Bfs} are:

l₁:=tr(C)

l₄ᵢ:=C:(i₀ ⊕i₀)

l_{Bfs}:=C:sym(f₀ ⊕s₀)

Here, C, f₀ and s₀ are the right Cauchy-Green tensor, and vectors for the local fiber and sheet directions, respectively. l is the determinant of the deformation gradient, and D=2/K with K being the Bulk modulus. The myofiber angles were assumed to change from 60 in the epicardium to −60 in the endocardium (Genet et al., 2014).

To account for the effects of injections on the passive material properties, the material definition was adjusted as follows:

$${ψ}_{dev} = \frac{\overline{a}}{2 b} {e}^{b ( {l}_{1} - 3 )} + {\sum }_{i = f , s} \frac{{\overline{a}}_{i}}{2 {b}_{i}} \{ {e}^{{{b}_{i} ( {l}_{4 i} - 1 )}^{2}} - 1 \} + \frac{{\overline{a}}_{fs}}{2 {b}_{fs}} \{ {e}^{{{b}_{f s} ( {l}_{s f s} )}^{2}} - 1 \}$$

$$with$$

$$\overline{a} = a [ {C}_{index} + ( 1 - {C}_{index} ) {P}_{scaling} ]$$

where, C_{index} is used to alter the stiffness of the tissue, and P_{scaling} is a constant that scales the passive response linearly. The regional influence of the collagenase injection is determined by C_{index}. Together, C_{index} and P_{scaling} enable smoothly varying stiffness throughout the tissue between regions that are or are not affected by injections.

The active stress was computed as follows (Guccione et al., 1993, Walker et al., 2005, and Genet et al., 2014):

$${T}_{0} = {T}_{max } \frac{{Ca}_{0}^{2}}{{Ca}_{0}^{2} + E {Ca}_{50}^{2}} {C}_{t}$$

where Tₘₐₓ is the isometric tension at the longest sarcomere length and highest calcium concentration, and

$${C}_{t} = \frac{1}{2} ( 1 - cos ω ) ,$$

$$ω = \{ \begin{matrix}π \frac{t}{{t}_{0}} when 0 \leq t \leq {t}_{0} \\ π \frac{t - {t}_{0} + {t}_{r}}{{t}_{r}} when {t}_{0} \leq t \leq {t}_{0} + {t}_{r} \\ 0 when t \geq {t}_{0} + {t}_{r}\end{matrix} , \\ {t}_{r} = ml + b \\ m , b = constant \\ EC {a}_{50} = \frac{( C {a}_{0} ) max}{\sqrt{exp [ B ( l - {l}_{0} ) ] - 1}} , l = {l}_{R} \sqrt{2 {E}_{ff} + 1}$$

where B is a constant, l₀ is the sarcomere length with no active stress, l_{R} is the sarcomere length with the stress-free condition, and E_{ff} is the Lagrangian strain in the fiber direction. The total stress was the sum of active and passive stresses.

The material constants a and b, the isotropic stiffness of the tissue, a_{f}, b_{f}, bₛ and c_{fs} and b_{fs} were determined according to the volume-pressure curves reported by Klotz et al (2006). The recorded end diastolic volume (EDV) and pressure (EDP) were used to reproduce the “Klotz” curve or end diastolic pressure-volume relationship (EDPVR). Then, the material constants were determined using an optimization process in which the error between the “Klotz” curve and computational EDPVR was minimized. This optimization was implemented through an in-house Python script which used the sequential least squares (SLSQP) algorithm (Jones, Oliphant et al. 2001), and ABAQUS as the forward solver.

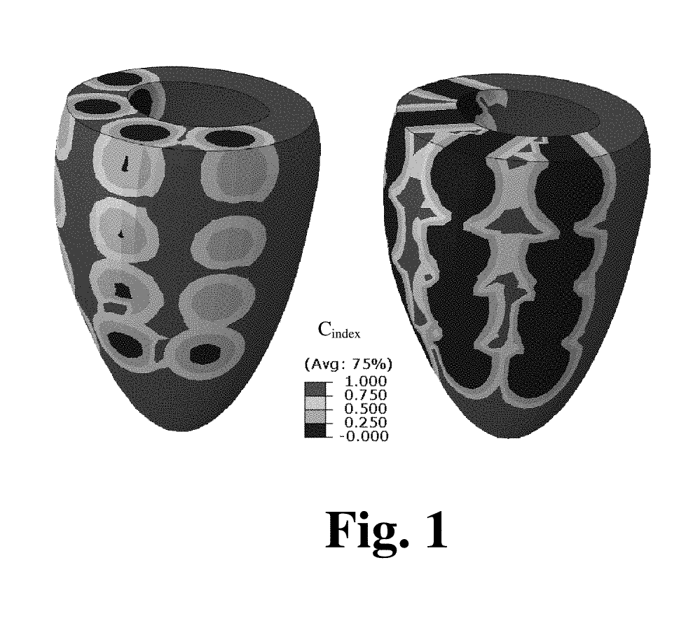

The untreated case refers to the LV 6 weeks after aortic banding with no injections. Based on previous studies (Weak et al., 2009, Wall et al., 2006), 1-16 injection sites were made in the LV free wall, to examine effects of injections in this region (FIG. 1). Additionally, to examine the effects of injections in the septum, an additional 12 injections were made in the septum. Two shapes of injections, cylindrical and spherical, were simulated. Cylindrical injections extended from the endocardium to the epicardium regions, whereas spherical regions were centered at each injection site. To examine the effects of injection stiffness, EDV was calculated for the 1-16 spherical injections with P_{scaling} altered at 0.01, 0.3, 0.5, 0.7, and 0.9. All the modeling was performed for the same animal LV.

### Results

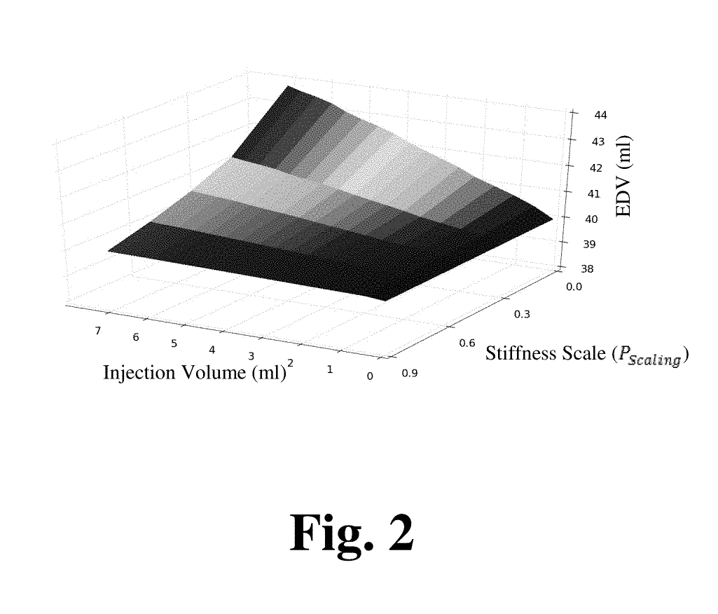

The EDV increased as the volume of injections increased. Moreover, the stiffness of the injections influenced EDV. When the stiffness of the injections decreased, the EDV increased (FIG. 2). The highest EDV (43.7 ml) was obtained with 16 injections (volume=8 ml) and lowest stiffness scale (P_{scaling}=0.01). Injection volume and stiffness scale strengthened the effect of each other. For a given injection volume, decreasing stiffness scale increases EDV, but an increase in EDV was more noticeable for higher volumes of injections. Likewise, for a given stiffness scale, higher injection volumes led to higher EDV, but increases in EDV were more noticeable for lower stiffness scale. In other words, the relation between injection volume, stiffness scale and EDV was coupled or nonlinear.

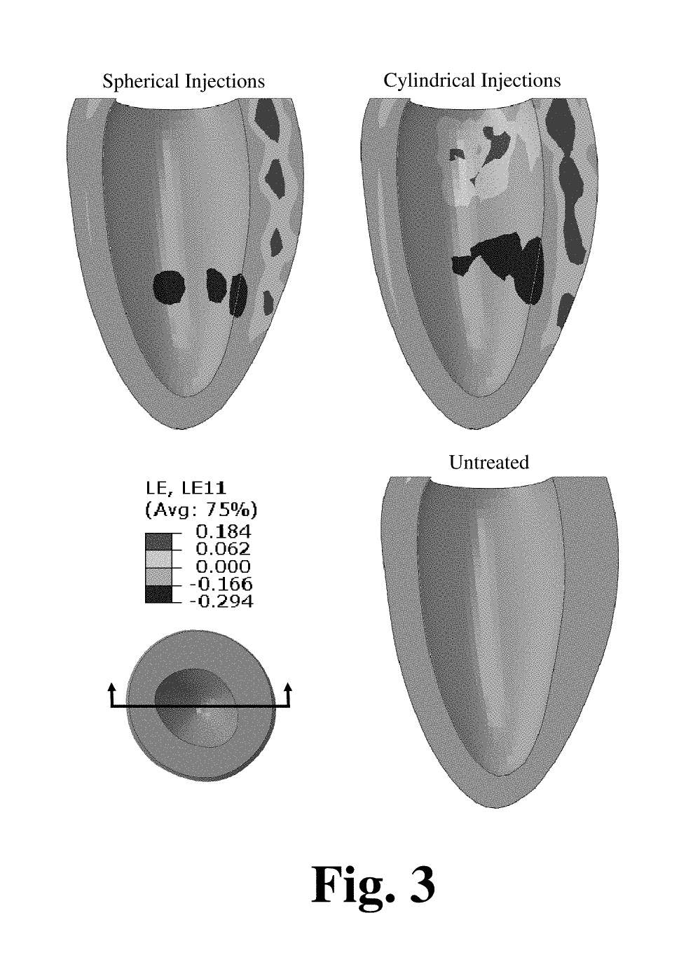

End systolic strain was perturbed due to injections. The LV mainly experienced shortening strain in the untreated case. Unlike the untreated case, lengthening strains were seen at the sites of injections located proximal to the base and/or epicardium. Compared to the untreated case, high shortening strains were seen at the injection sites within the endocardium and close to the apex (FIG. 3). At end systole, the injection sites did not show noticeable distortions compared to the surrounding tissue.

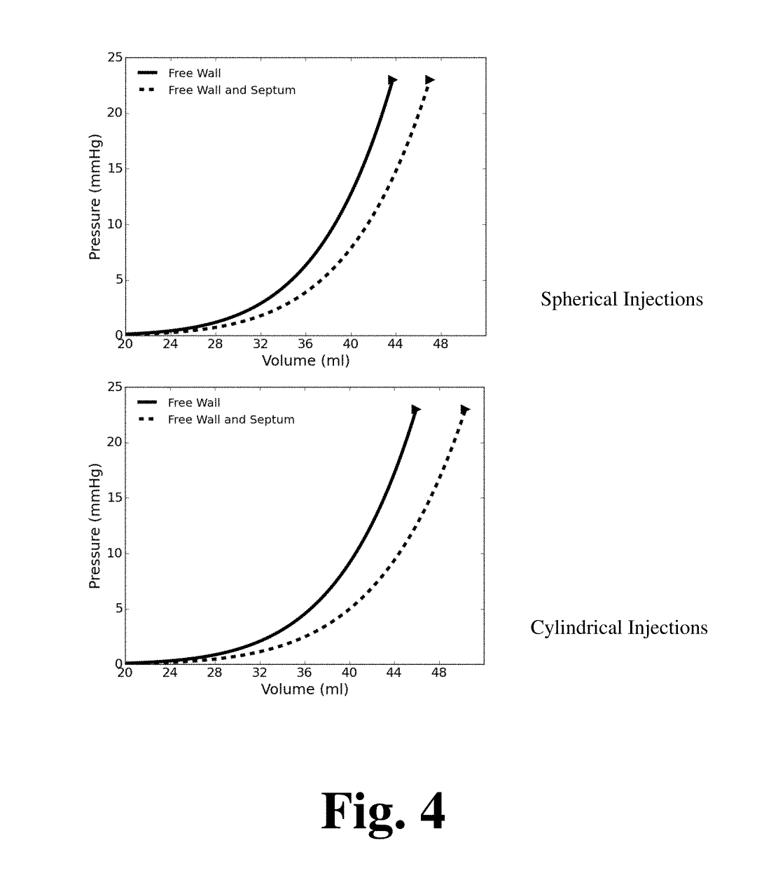

The EDPVR shifted toward higher EDVs when the injections were implemented in both septum and free wall than when they were only within the free wall (FIG. 4). Both spherical and cylindrical injections led to higher EDV when implemented in the free wall in addition to the septum. However, cylindrical injections led to higher EDVs. At EDP=23 mmHg, the cylindrical injections and spherical injections in the free wall and septum led to EDV=50.4 and 47.0 ml, respectively (Table 1, below).

TABLE 1
The EDV for normal base-line, untreated pressure overload, and
injection treated states for the animal studied. The injection volume
represents the region where C_{index} = 0 (the dark blue (darkest)
region in FIG. 1).

| Injection shape | Injection Volume (ml) | EDV (ml) |
|---|---|---|
| Healthy Base-line | NA | 41.9 |
| Untreated Pressure Overload | NA | 40.0 |
| Spherical Injections | | |
| Free wall | | |
| Mid-wall | 7.0 | 43.7 |
| Endocardium | 4.2 | 43.9 |
| Epicardium | 3.8 | 41.2 |
| Free wall and septum | 11.8 | 47.0 |
| Cylindrical Injections | | |
| Free wall | 8.0 | 46.0 |
| Free wall and septum | 11.1 | 50.4 |

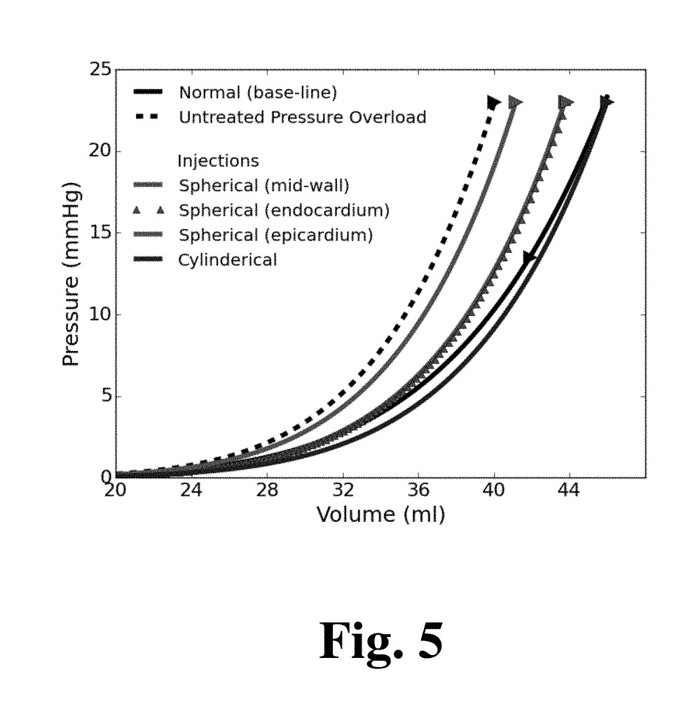

The injections in the free wall shifted the EDPVR toward higher EDVs compared to the untreated case (FIG. 5). The cylindrical injections had greater effects on increasing EDV compared to spherical injection. At EDP=23 mmHg, the EDV was 46.0 ml for cylindrical injections. At the same EDP, the EDV for spherical injections implemented in mid-wall, endocardium and epicardium was 43.7, 43.9 and 41.2 ml respectively (Table 1).

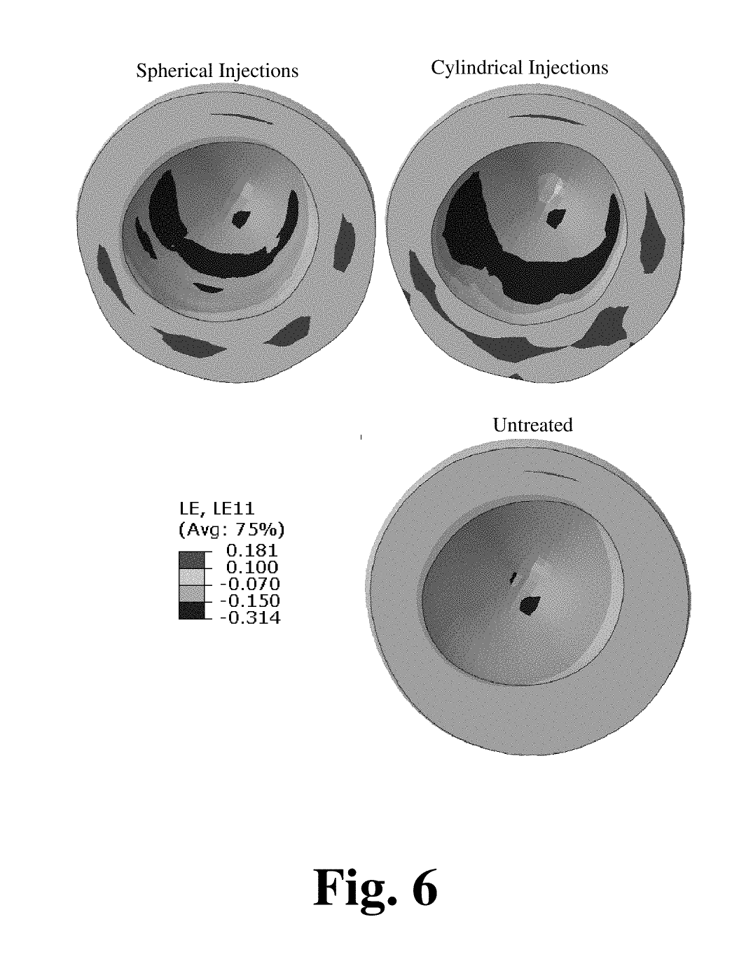

At the injected regions, the subendocardial region experienced higher strains compared to the untreated case (FIG. 6). Similarly to end systolic strains, ED strains increased at the injection sites, compared to the untreated case. The injection sites near the apex and within the endocardium experienced high shortening strains, whereas the injections sites proximal to the base and epicardium experienced high lengthening strains.

### Discussion

The present disclosure includes disclosure of the first FE modeling study of a treatment for HPpEF that reduces the stiffness of the diastolic myocardium or extracellular matrix at strategic locations. The findings provided herein demonstrate that virtual injections of collagenase would be (is) an effective treatment for HFpEF. In at least one injection method of the present disclosure, collagenase exits the needle as the needle is moved from endocardium to epicardium (in the case of an endocardial catheter), resulting in cylindrically-shaped affected regions, versus only have collagenase exit the needle when it is placed at mid-wall, resulting in spherically-shaped affected regions. Specifically, with 8.0 ml of cylindrical injections in only the free wall and with EDP=23 mmHg, EDV increased by 15.0% compared to the untreated case (FIG. 1, Table 1). With 11.1 ml of cylindrical injections in both the free wall and the septum, EDV increased by 26.0% (FIG. 4, Table 1).

Abnormally high LV stiffness is suggested as a factor for the development of HFpEF, which can be seen in the EDPVR (Lam et al., 2007, Zile et al., 2004). With injections in the free wall, the stiff LV EDPVR was noticeably de-stiffened by shifting toward higher EDVs, and the LV EDPVR further de-stiffened with injections in both the free wall and the septum (FIGS. 4 and 5). The pattern of injections, the site of injections, the injection volume, and stiffness of the injections profoundly affected the effectiveness of injection treatment (FIGS. 2, 4 and 5). The end systolic myofiber strain was also increased by injection treatment (FIG. 3).

As a result of injection treatment, the LV was de-stiffened by shifting the EDPVR toward higher EDVs, which consequently recovered the LV mechanics. Higher expansion during diastole led to a larger EDV, and the LV wall became less resistant to the filling volume, as shown in EDPVRs (FIG. 4). Cylindrical injections that spread all the way from endocardium to epicardium were more effective in increasing EDV and improving the LV EDPVR (FIGS. 4 and 5, Table 1). Therefore, in terms of EDV and EDPVR, injections that spread in the transmural direction would be better than injections that lead to spherical regions of affected myocardium. Moreover, the endocardium has a key role in injection treatment. Although the injection volume was less for injections in the endocardium than in the mid-wall, they led to a higher increase in EDV (FIG. 5, Table 1).

Although the free wall is more accessible for injection than the septum, the LV mechanics would be improved significantly if the injections were made in both the free wall and the septum (FIG. 4). The results of the studies reference herein identify that at EDP=23 mmHg, with 8.0 ml injections only in the free wall, EDV increased by 15%, but that with 11.1 ml of injections in both the free wall and the septum, EDV increased by 26%, compared to the untreated case (Table 1). This finding confirms that the more sites of injections in the LV wall, the better treatment outcomes could be, at least for EDVs in the EDPVR.

The endocardial surface experienced high strains in the injection regions, but noticeable distortions in injected regions, compared to the surrounding area (FIG. 6), were not identified. Although the injections did not lead to noticeable distortions in the injected sites, they noticeably altered the global mechanics of the LV, including EDV and EDPVR. As long as the injections target the ECM, they are not expected to affect the functionality of the fibers. It is particularly crucial to avoid any damage to the fibers in the subendocardial region because unlike the epicardial region, it plays a key role in LV shortening and is more vulnerable to ischemia (Sabbah et al., 1981, Algranati et al., 2011).

This study referenced herein focused on effects of LV wall de-stiffening on the mechanics of LV, using injection treatment. As collagenase is the major component of the ECM, collagenase was used as the exemplary injection material, and other enzymes that target proteoglycans and ground substance may be better options for injection treatment of HFpEF.

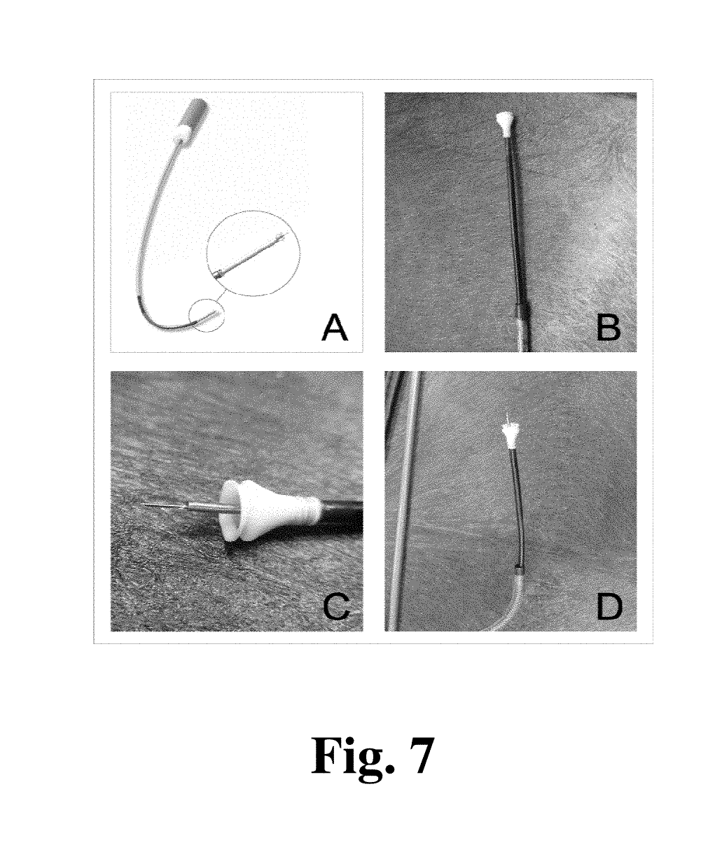

Development of a novel catheter used for injection treatment makes it possible to implement the methods of the present disclosure more effectively. In particular, and for example, injections in the septum in addition to the free wall can lead to more effective outcomes in LV diastolic filling and mechanics (FIG. 4, Table 1). In addition, injections into the subendocardium are more effective than injections at mid-myocardium or in the subepicardium (FIG. 5, Table 1). A novel catheter has been developed that provides access to the pericardial space percutaneously (Kassab et al., 2010; Sulkin et al., 2016, FIG. 7), which can be used to make injections into the septum as well as the endocardium. Moreover, the cylindrical injections could be made by controlled withdrawal of this catheter during injection in the LV wall.

In the studies contained within the present disclosure, experimentally recorded LV volume and pressure were used to calculate mechanical properties in an untreated LV. In addition, measuring LV pressure and volume to quantify LV mechanical properties at specific time intervals after injection can be performed, and these long-term changes can be incorporated into various models identified herein. Assessments of alterations in LV mechanical properties compared to normal, diseased, and treated states at various time points can also be determined using experimental data obtained consistent with the present disclosure.

The results of the studies performed in connection with the present disclosure pertain to short-term effects of injections on LV mechanics. The effects of injections on LV mechanics have key roles in optimal planning for injection treatment (Wall et al. 2006). As such, the present disclosure includes disclosure of therapeutically altering passive mechanics within the LV to enable better LV filling in a subject with abnormally stiff myocardium.

The present disclosure includes the first FE modeling study of a treatment for HPpEF that reduces the stiffness of the diastolic myocardium or extracellular matrix at strategic locations. The injection treatment can be an efficient treatment for HFpEF, as disclosed herein, which would be much less expensive and less risky compared to current options. Using physics-based modeling, the study referenced in the present disclosure demonstrates how the optimal stiffness, pattern, and volume can be planned for injection treatment for HFpEF.

FE modeling is an optimal method that provides key insights about the effects of injections on the EDPVR. FE modeling for injection treatment for heart failure with reduced ejection fraction has previously been used, which effectively led to preclinical studies (Wall et al., 2006). Using injection treatment, the EDPVR can be de-stiffened, and more importantly, using computational modeling, the level of de-stiffening can be planned by adjusting injection specifications. Therefore, a virtual set of injections that result in greatest increases in EDVs in the EDPVR can be identified and administered as desired.

While various embodiments of methods to treat heart failure the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCE LIST

Algranati D, Kassab G S, Lanir Y. Why is the subendocardium more vulnerable to ischemia? A new paradigm. Am J Physiol Heart Circ Physiol. 2011;300:H1090-100.

Baillargeon B, Costa I, Leach J R, Lee L C, Genet M, Toutain A, et al. Human cardiac function simulator for the optimal design of a novel annuloplasty ring with a sub-valvular element for correction of ischemic mitral regurgitation. Cardiovasc Eng Technol. 2015;6:105-16.

Baillargeon B, Rebelo N, Fox D D, Taylor R L, and Kuhl E, The Living Heart Project: A robust and integrative simulator for human heart function, Eur J Mech A Solids. 2014; 48: 38-47.

Benjamin E J, Blaha M J, Chiuve S E, Cushman M, Das S R, Deo R, et al. American Heart Association Statistics Committee and Stroke Statistics Subcommittee. Heart disease and stroke statistics-2017 update: a report from the American Heart Association. Circulation. 2017;135:e146-e603.

Bhuiyan T and Maurer M S. Heart Failure with Preserved Ejection Fraction: Persistent Diagnosis, Therapeutic Enigma, Curr Cardiovasc Risk Rep. 2011;5: 440-449.

Bursi F, Weston S A, Redfield M M, Jacobsen S J, Pakhomov S, Nkomo V T, et al. Systolic and diastolic heart failure in the community. JAMA. 2006;296:2209-16.

Genet M, Lee L C, Nguyen R, Haraldsson H, Acevedo-Bolton G, Zhang Z, et al. Distribution of normal human LV myofiber stress at end diastole and end systole: a target for in silico design of heart failure treatments. J Appl Physiol. 2014;117:142-152.

Göktepe S, Acharya S N S, Wong J, Kuhl E. Computational modeling of passive myocardium. International Journal for Numerical Methods in Biomedical Engineering. 2011;27:1-12.

Guccione J M, McCulloch A D. Mechanics of active contraction in cardiac muscle. I. Constitutive relations for fiber stress that describe deactivation. J Biomech Eng. 1993;115:72-81.

Guccione J M, McCulloch A D, Waldman L K. Passive material properties of intact ventricular myocardium determined from a cylindrical model. J Biomech Eng. 1991;113:42-55.

Heidenreich P A, Albert N M, Allen L A, Bluemke D A, Butler J, Fonarow G C, et al. Forecasting the impact of heart failure in the United States: A policy statement from the American Heart Association. Circ Heart Fail. 2013;6:606-619.

Holzapfel G A, Ogden R W. Constitutive modelling of passive myocardium: a structurally based framework for material characterization. Philos Trans A Math Phys Eng Sci. 2009;367:3445-75.

Jones E, Oliphant T and Peterson P (2001). SciPy: Open source scientific tools for Python.

Kassab G S, Svendsen M, Combs W, Choy J S, Berbari E J, Navia J A. A transatrial pericardial access: lead placement as proof of concept. Am J Physiol Heart Circ Physiol. 2010;298:H287-93.

Klotz S, Hay I, Dickstein M L, Yi G H, Wang J, Maurer M S, et al. Single-beat estimation of end-diastolic pressure-volume relationship: a novel method with potential for noninvasive application. Am J Physiol Heart Circ Physiol. 2006; 291: H403H412.

Lam C S, Roger V L, Rodeheffer R J, Bursi F, Borlaug B A, Ommen S R, et al. Cardiac structure and ventricular-vascular function in persons with heart failure and preserved ejection fraction from Olmsted County, Minnesota. Circulation. 2007;115:1982-90.

Lee L C, Wall S T, Klepach D, Ge L, Zhang Z, Lee R J, et al. Algisyl-LVR™ with coronary artery bypass grafting reduces left ventricular wall stress and improves function in the failing human heart. Int J Cardiol. 2013;168:2022-8.

Lee R J, Hinson A, Bauernschmitt R, Matschke K, Fang Q, Mann D L, et al. The feasibility and safety of Algisyl-LVR™ as a method of left ventricular augmentation in patients with dilated cardiomyopathy: initial first in man clinical results. Int J Cardiol. 2015;199:18-24.

Liu Y, Haddad T, Dwivedi G. Heart failure with preserved ejection fraction: current understanding and emerging concepts. Curr Opin Cardiol. 2013;28:187-96.

Mann D L, Lee R J, Coats A J, Neagoe G, Dragomir D, Pusineri E, et al. One-year follow-up results from AUGMENT-HF: a multicentre randomized controlled clinical trial of the efficacy of left ventricular augmentation with Algisyl in the treatment of heart failure. Eur J Heart Fail. 2016;18:314-25.

Owan T E, Hodge D O, Herges R M, Jacobsen S J, Roger V L, Redfield M M. Trends in prevalence and outcome of heart failure with preserved ejection fraction. N Engl J Med. 2006: 20;355:251-9.

Sabbah H N, Marzilli M, Stein P D. The relative role of subendocardium and subepicardium in left ventricular mechanics. Am J Physiol. 1981;240:H920-6.

Sack K L, Baillargeon B, Acevedo-Bolton G, Genet M, Rebelo N, Kuhl E, et al. Partial LVAD restores ventricular outputs and normalizes LV but not RV stress distributions in the acutely failing heart in silico. Int J Artif Organs. 2016;39:421-430.

Steinberg B A, Zhao X, Heidenreich P A, Peterson E D, Bhatt D L, Cannon C P, et al. Trends in patients hospitalized with heart failure and preserved left ventricular ejection fraction: prevalence, therapies, and outcomes. Circulation. 2012;126:65-75.

Sulkin M S, Berwick Z C, Hermiller J B, Navia J A, Kassab G S. Suction catheter for enhanced control and accuracy of transseptal access. EuroIntervention. 2016;12:1534-1541.

Vasan R S, Benjamin E J, Levy D. Prevalence, clinical features and prognosis of diastolic heart failure: an epidemiologic perspective. J Am Coll Cardiol. 1995;26:1565-74.

Walker J C, Ratcliffe M B, Zhang P, Wallace A W, Fata B, Hsu E W, Saloner D, Guccione J M. MRI-based finite-element analysis of left ventricular aneurysm. Am J Physiol Heart Circ Physiol. 2005;289:H692H700.

Wall S T, Walker J C, Healy K E, Ratcliffe M B, Guccione J M. Theoretical impact of the injection of material into the myocardium: a finite element model simulation. Circulation. 2006;114:2627-35.

Wenk J F, Wall S T, Peterson R C, Helgerson S L, Sabbah H N, Burger M, et al. A method for automatically optimizing medical devices for treating heart failure: designing polymeric injection patterns. J Biomech Eng. 2009;13:121011.

Zile M R, Baicu C F, Gaasch W H. Diastolic heart failure—abnormalities in active relaxation and passive stiffness of the left ventricle. N Engl J Med. 2004;350:1953-9.

1. A method, comprising the step of:
injecting a material into a cardiac tissue that targets an extracellular matrix of the cardiac tissue to treat heart failure.

2. The method of claim 1, wherein the material comprises an enzyme, and wherein the injecting step is performed to inject the enzyme into the cardiac tissue.

3. The method of claim 2, wherein the cardiac tissue comprises a left ventricle wall, and wherein the injecting step is performed to inject the enzyme into the left ventricle wall.

4. The method of claim 2, wherein the enzyme comprises collagenase, and wherein the injecting step is performed to inject the collagenase into the cardiac tissue.

5. The method of claim 2, wherein the enzyme comprises at least one enzyme that targets proteoglycans and ground substance, and wherein the injecting step is performed to inject the at least one enzyme that targets proteoglycans and ground substance into the cardiac tissue.

6. The method of claim 1, wherein the extracellular matrix is stiff, and wherein the injecting step is performed to inject the material into the cardiac tissue that targets the stiff extracellular matrix.

7. The method of claim 1, wherein the step of injecting reduces a stiffness of the extracellular matrix of the cardiac tissue.

8. The method of claim 1, wherein the step of injecting reduces a stiffness of a diastolic myocardium of the cardiac tissue.

9. The method of claim 1, wherein the heart failure is heart failure with preserved injection fraction (HFpEF), and wherein the step of injecting is performed to treat HFpEF.

10. The method of claim 1, wherein the step of injecting is performed so that material injected into the cardiac tissue has a spherical shape.

11. The method of claim 1, wherein the step of injecting is performed so that material injected into the cardiac tissue has a cylindrical shape.

12. The method of claim 11, wherein the step of injecting is performed so that the cylindrical shape of the material injected into the cardiac tissue extends between an endocardium and an epicardium of the cardiac tissue.

13. The method of claim 1, wherein the step of injecting is performed to inject the material at a first injection site and at a second injection site of the cardiac tissue.

14. The method of treating heart failure of claim 13, wherein the first injection site is selected from the group consisting of a mid-wall, an epicardium, and an endocardium.

15. The method of claim 1, wherein the step of injecting is performed to inject between 7.0 mL and 11.8 mL of the material into the cardiac tissue.

16. A method, comprising the step of:
injecting a material comprising an enzyme into a left ventricle of a cardiac tissue, the material targeting an extracellular matrix of the cardiac tissue to treat heart failure with preserved injection fraction (HFpEF).

17. The method of claim 16, wherein the step of injecting is performed so to inject the material between once and sixteen times into a free wall of the left ventricle.

18. The method of claim 16, further comprising the step of:
injecting the material into a septum of the cardiac tissue between once and twelve times.

19. A method of using a minimally invasive injection of an extracellular matrix targeting material at a location in a cardiac tissue having an abnormally stiff extracellular matrix as an effective treatment for heart failure with preserved injection fraction (HFpEF), the method comprising the steps of:
percutaneously accessing a pericardial space of a heart; and
injecting a free wall of the cardiac tissue with the extracellular matrix targeting material, the extracellular matrix targeting material comprising collagenase or another enzyme that targets proteoglycans and ground substance.

20. The method of claim 19, further comprising the steps of:
first determining an end diastolic volume (EDV) of the heart before the step of injecting is performed; and
second determining an end diastolic volume (EDV) of the heart after the step of injecting is performed, wherein the EDV of the heart after the step of injecting is higher than the EDV of the heart before the step of injecting.
